(19) United States
(12) Patent Application Publication
(10) Pub. No.: US 2026/0314963 A1
(43) Pub. Date: Oct. 15, 2026

(54) DETECTION DEVICE

(71) Applicant: Japan Display Inc., Tokyo (JP)

(72) Inventor: Yoshitaka KOJIMA, Tokyo (JP)

(21) Appl. No.: 19/464,265

(22) Filed: Jan. 29, 2026

Related U.S. Application Data
(63) Continuation of application No. PCT/JP2024/027097, filed on Jul. 30, 2024.

(30) Foreign Application Priority Data
Aug. 17, 2023 (JP) 2023-132821

(51) Int. Cl. A61B 5/1455; A61B 5/00; A61B 5/024; H10H 20/855; H10H 29/24; H10K 30/60; H10K 30/81; H10K 39/00; H10K 50/86; H10K 59/95

(52) U.S. Cl. CPC A61B 5/14552; A61B 5/02416; A61B 5/6826; H10H 20/855; H10H 29/24; H10K 30/60; H10K 30/81; H10K 39/621; H10K 50/86; H10K 59/95

(57) ABSTRACT

According to an aspect, a detection device includes: a substrate; a light source configured to emit light to an object to be detected; and an organic photodiode in which a lower electrode, a lower buffer layer, an active layer, an upper buffer layer, and an upper electrode are stacked in the order as listed. The organic photodiode is provided on the substrate. An opening provided so as to penetrate the organic photodiode exposes a surface of the substrate. The light source is provided on the substrate in the opening.

### CROSS-REFERENCE TO RELATED APPLICATION

This application claims the benefit of priority from Japanese Patent Application No. 2023-132821 filed on Aug. 17, 2023 and International Patent Application No. PCT/JP2024/027097 filed on Jul. 30, 2024, the entire contents of which are incorporated herein by reference.

### BACKGROUND

### 1. Technical Field

What is disclosed herein relates to a detection device.

### 2. Description of the Related Art

Optical sensors capable of measuring pulse waves at a fingertip are known (for example, Japanese Patent Application Laid-open Publication No. 2015-134177 (JP-A-2015-134177)). In such an optical sensor, light emitted from a light-emitter (light source) is scattered and reflected by an object to be detected and enters a light receiver (photodiode). Thus, the light receiver detects biometric information on the object to be detected.

In the optical sensor described in JP-A-2015-134177, the light source and the photodiodes are provided on the same light-transmitting member and arranged in a horizontal row. This configuration causes a mounting area for components to be larger, which makes it harder to reduce the overall device size. In addition, the amount of light that can be absorbed by the photodiodes when the photodiodes are irradiated with light becomes limited, which may reduce the light use efficiency.

For the foregoing reasons, there is a need for a detection device that can reduce the size of the area for mounting the light source and the components of photodiodes and increase the light use efficiency.

### SUMMARY

According to an aspect, a detection device includes: a substrate; a light source configured to emit light to an object to be detected; and an organic photodiode in which a lower electrode, a lower buffer layer, an active layer, an upper buffer layer, and an upper electrode are stacked in the order as listed. The organic photodiode is provided on the substrate. An opening provided so as to penetrate the organic photodiode exposes a surface of the substrate. The light source is provided on the substrate in the opening.

### BRIEF DESCRIPTION OF THE DRAWINGS

FIG. 1 is a schematic view illustrating an exemplary external appearance when a state of a finger accommodated inside a detection device according to a first embodiment is viewed from a lateral side of a housing;

FIG. 2 is a schematic sectional view of a section taken along II-II' illustrated in FIG. 1;

FIG. 3 is a schematic view illustrating a configuration example of an optical sensor and light sources of the detection device illustrated in FIG. 2;

FIG. 4 is a plan view illustrating the lowermost layer of a substrate according to the first embodiment;

FIG. 5 is a schematic sectional view of a section taken along V-V' in FIG. 3;

FIG. 6 is a schematic sectional view of a section taken along VI-VI' in FIG. 3;

FIG. 7 is a circuit diagram illustrating a configuration example of the detection device according to the first embodiment;

FIG. 8 is a flowchart illustrating an example of a process to output a blood oxygen saturation level in the first embodiment;

FIG. 9 is a timing waveform diagram illustrating an operation example using a first light source and a second light source of the detection circuit according to the first embodiment;

FIG. 10 is a plan view illustrating a pulse wave detection area when using the first light source and the second light source of the detection device according to the first embodiment;

FIG. 11 is the circuit diagram illustrating a configuration example of the detection device according to the first embodiment;

FIG. 12 is a flowchart illustrating exemplary processing related to output of a heart rate in the first embodiment;

FIG. 13 is a timing waveform diagram illustrating an operation example using third light sources of the detection circuit according to the first embodiment;

FIG. 14 is a plan view illustrating pulse wave detection areas when using the third light sources of the detection device according to the first embodiment; and

FIG. 15 is a plan view illustrating pulse wave detection areas when using the third light sources of a detection device according to a comparative example.

### DETAILED DESCRIPTION

The following describes a mode (embodiment) for carrying out the present disclosure in detail with reference to the drawings. The present disclosure is not limited to the description of the embodiment given below. Components described below include those that are easily conceivable by those skilled in the art or those that are substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present specification and claims, in expressing an aspect of disposing another structure on or above a certain structure, a case of simply expressing "on" includes both a case of disposing the other structure immediately on the certain structure so as to contact the certain structure and a case of disposing the other structure above the certain structure with still another structure interposed therebetween, unless otherwise specified.

### First Embodiment

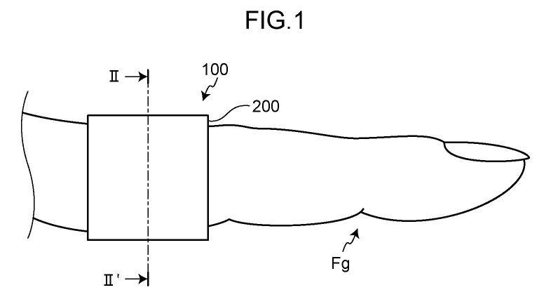

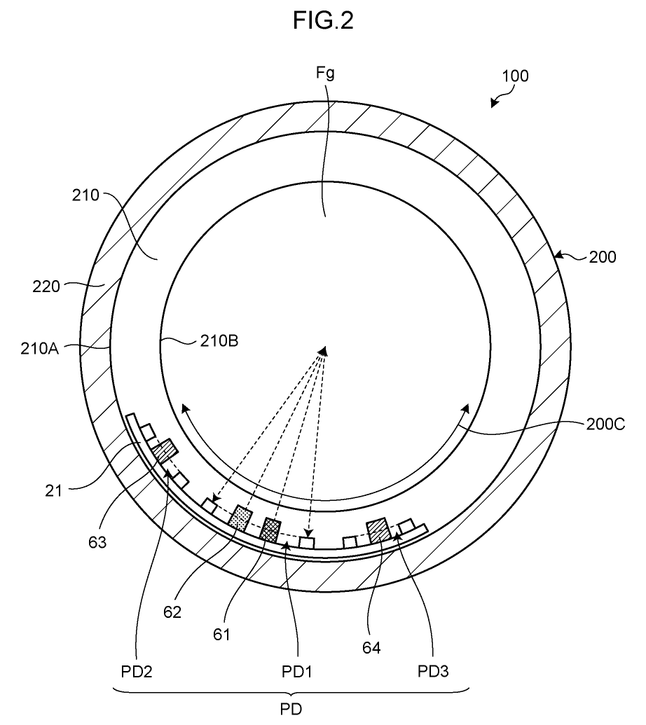

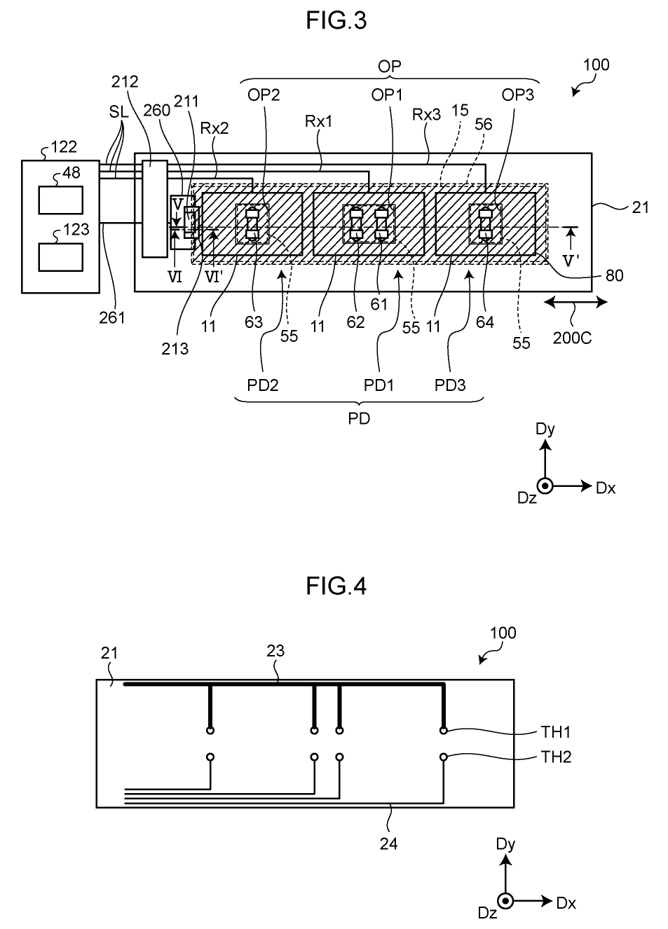

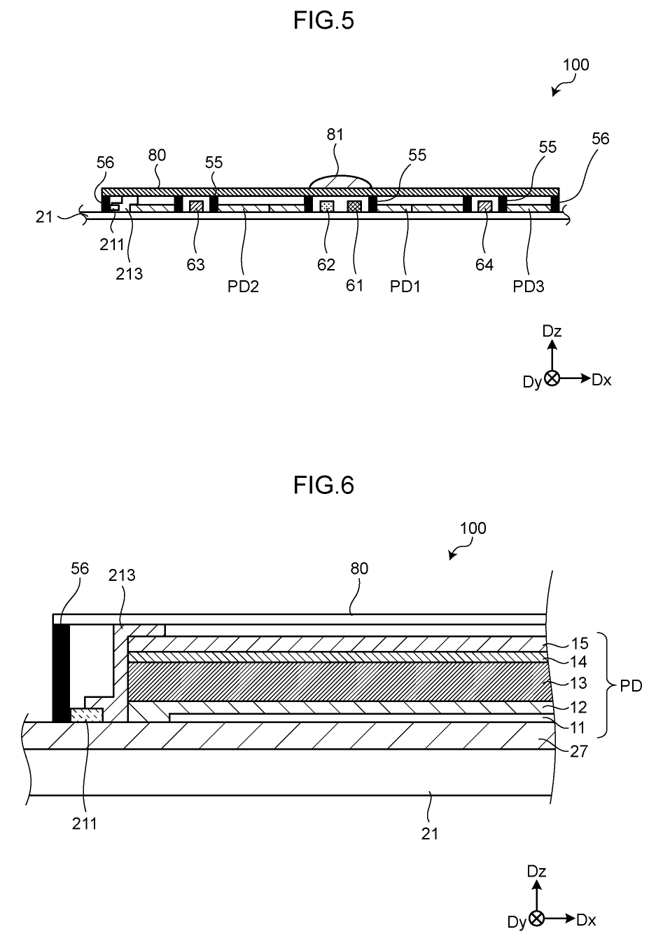

FIG. 1 is a schematic view illustrating an exemplary external appearance when a state of a finger accommodated inside a detection device according to a first embodiment is viewed from a lateral side of a housing. FIG. 2 is a schematic sectional view of a section taken along II-II' illustrated in FIG. 1. FIG. 3 is a schematic view illustrating a configuration example of an optical sensor and light sources of the detection device illustrated in FIG. 1. FIG. 4 is a plan view illustrating a substrate according to the first embodiment. FIG. 5 is a schematic sectional view of a section taken along V-V' illustrated in FIG. 3. FIG. 6 is a schematic sectional view of a section taken along VI-VI' illustrated in FIG. 3. FIG. 2 illustrates only the basic configuration of the detection device according to the first embodiment and does not illustrate the other configurations.

A detection device 100 illustrated in FIG. 1 is a finger ring-shaped device that can be worn on and removed from a human body and is worn on an object to be detected Fg of the human body. Examples of the object to be detected Fg include a thumb, an index finger, a middle finger, a ring finger, and a little finger. The human body is a person to be authenticated whose identity is to be verified by the detection device 100. The detection device 100 can detect biometric information on a living body from the object to be detected Fg wearing the detection device 100. The object to be detected Fg is an example of a measurement target. The measurement target is the living body or a part of the living body and is an object to be measured. The detection device 100 is formed as a finger ring or a wristband so as to be easily carried by a user. In the following description, the detection device 100 is assumed to be used as a finger ring.

As illustrated in FIG. 2, the detection device 100 includes a housing 200, a substrate 21, a first light source 61, a second light source 62, third light sources 63 and 64, and an optical sensor PD. The optical sensors PD include a first optical sensor PD1, a second optical sensor PD2, and a third optical sensor PD3. The detection device 100 is a device that includes a battery (not illustrated) inside the housing 200 and is operated by power of the battery.

As illustrated in FIGS. 3 and 5, the optical sensors PD are provided on the substrate 21. An opening OP provided so as to penetrate the optical sensor PD exposes a surface of the substrate 21. As illustrated in FIG. 3, the first optical sensor PD1, the second optical sensor PD2, and the third optical sensor PD3 are configured with respective lower electrodes 11 arranged along a circumferential direction 200C. A plurality of the openings OP are provided so as to penetrate the respective lower electrodes 11. The openings OP include a first opening OP1, a second opening OP2, and a third opening OP3. The first light source 61, the second light source 62, and the third light sources 63 and 64 are each provided on the substrate 21. The first light source 61 and the second light source 62 are provided in the circumferential direction 200C along a first direction Dx in the first opening OP1. The second and the third openings OP2 and OP3 are provided with the third light sources 63 and 64, respectively. The first opening OP1 is provided between the second and the third openings OP2 and OP3. A pair of the first and second light sources 61 and 62, the third light source 63, and the third light source 64 are each surrounded by a corresponding one of the lower electrodes 11.

As illustrated in FIGS. 1 and 2, the housing 200 is a wearable member that is formed in a ring shape (annular shape) wearable on the object to be detected Fg and is worn on the living body. In the example illustrated in FIG. 2, the housing 200 includes a first housing 210 and a second housing 220. The first housing 210 is integrated with the second housing 220 to form the housing 200 into the ring shape. The first housing 210 is a member that contacts the human body on which the housing 200 is worn. The first housing 210 accommodates therein the first light source 61, the second light source 62, the third light sources 63 and 64, the optical sensors PD, and other components. The first housing 210 is formed into a ring shape using a housing material, such as a light-transmitting synthetic resin or silicon. The second housing 220 has a surface of the housing 200 that covers an outer peripheral surface 210A of the first housing 210. The second housing 220 is formed into a ring shape using a member of, for example, a metal or a non-light-transmitting synthetic resin. The first housing 210 of the housing 200 accommodates therein the substrate 21 on which the first light source 61, the second light source 62, the third light sources 63 and 64, the optical sensors PD, and other components are mounted. The substrate 21 is accommodated in the housing 200, for example, by being formed into a ring shape and, in that state, filling a surrounding area thereof with a filling material in a mold to form the housing 200.

As illustrated in FIG. 3, the substrate 21 is formed into a deformable band shape. The substrate 21 is a base member formed by bonding two stacked layers. The substrate 21 is an insulating substrate and is, for example, a light-transmitting flexible printed circuit (FPC) board. The first light source 61, the second light source 62, the third light sources 63 and 64, and other components are mounted on a surface of the uppermost layer of the substrate 21. The substrate 21 electrically couples a control circuit 122 to the first light source 61, the second light source 62, the third light sources 63 and 64, the optical sensors PD, and other components.

In the following description, the first direction Dx is one direction in a plane parallel to the first substrate 21. A second direction Dy is one direction in the plane parallel to the substrate 21 and is a direction orthogonal to the first direction Dx. The second direction Dy may non-orthogonally intersect the first direction Dx. A third direction Dz is a direction orthogonal to the first direction Dx and the second direction Dy. The third direction Dz is a direction normal to the first substrate 21. The term "plan view" refers to a positional relation as viewed from a direction orthogonal to the substrate 21.

In the present embodiment, as illustrated in FIG. 2, the substrate 21 is accommodated in the housing 200 such that the surface of the substrate 21 provided with the optical sensors PD, the first light source 61, the second light source 62, and the third light sources 63 and 64 face an inner peripheral surface 210B of the housing 200. When the substrate 21 has a light-transmitting property, the optical sensors PD, the first light source 61, the second light source 62, and the third light sources 63 and 64 may be mounted on the back surface opposite the surface. In this case, the first light source 61, the second light source 62, and the third light sources 63 and 64 only need to be arranged such that light is emitted toward the substrate 21 and then light transmitted through the substrate 21 exits outward from the inner peripheral surface 210B of the housing 200.

As illustrated in FIG. 2, the first light source 61, the second light source 62, and the third light sources 63 and 64 are provided in the housing 200 and are capable of emitting light toward the object to be detected Fg wearing the ring-shaped housing 200. For example, inorganic light-emitting diodes (LEDs) or organic electroluminescent (EL) diodes (organic light-emitting diodes (OLEDs)) are used as the first light source 61, the second light source 62, and the third light sources 63 and 64. The first light source 61, the second light source 62, and the third light sources 63 and 64 emit light having predetermined wavelengths. In the present embodiment, the first light source 61 includes a light source capable of emitting red light. The second light source 62 includes a light source capable of emitting infrared light. The third light sources 63 and 64 include a light source capable of emitting green light.

As illustrated in FIGS. 2 and 3, the light emitted from the first and the second light sources 61 and 62 and reflected by the object to be detected Fg enters the first optical sensor PD1. The light emitted from the third light source 63 and reflected on the surface of the object to be detected Fg enters the second optical sensor PD2. The light emitted from the third light source 64 and reflected on the surface of the object to be detected Fg enters the third optical sensor PD3. Thus, the detection device 100 can detect information on a living body in the object to be detected Fg. Examples of the information on the living body include, but are not limited to, pulse waves and pulsation of the finger or a palm. That is, the detection device 100 may be configured as a vein detection device that detects biometric information on veins or the like.

The optical sensors PD are provided in the first housing 210 of the housing 200 and are capable of detecting light incident from light emitting sides of the first light source 61, the second light source 62, and the third light sources 63 and 64. The light emitting sides of the first light source 61, the second light source 62, and the third light sources 63 and 64 are the inside of the ring-shaped housing 200, and are sides of light sources that irradiate the object to be detected Fg serving as the measurement target wearing the housing 200. The optical sensor PD is an organic photodiode (OPD). By being mounted on the substrate 21, the optical sensors PD are provided in the housing 200 so as to be arranged with the first light source 61, the second light source 62, and the third light sources 63 and 64 in the circumferential direction 200C (first direction Dx) of the housing 200.

The first optical sensor PD1, the second optical sensor PD2, and the third optical sensor PD3 are electrically coupled to the uppermost layer side of the substrate 21. The first optical sensor PD1, the second optical sensor PD2, and the third optical sensor PD3 have a multilayered configuration including the lower electrodes 11 and one upper electrode 15. The upper electrode 15 covers three lower electrodes 11 in plan view.

As illustrated in FIG. 3, one end sides of first wiring lines Rx1, Rx2, and Rx3 provided on the surface of the uppermost layer of the substrate 21 are coupled to a detection circuit 48 included in the control circuit 122 via a plurality of signal lines SL. The other end sides of the first wiring lines Rx1, Rx2, and Rx3 are electrically coupled to the lower electrodes 11. The first wiring lines Rx1, Rx2, and Rx3 are formed, for example, of metal wiring, and are formed of material having better conductivity than the lower electrodes 11 of the optical sensor PD. The first wiring lines Rx1, Rx2, and Rx3 are formed, for example, of light-transmitting conductive material such as indium tin oxide (ITO).

As illustrated in FIGS. 3 and 6, the detection device 100 includes a power supply electrode 211 extending along the second direction Dy. The power supply electrode 211 is electrically coupled to a coupler 212 via a conductive material member 213, and a sensor power supply signal (sensor power supply voltage) is supplied from a power supply circuit 123 via the coupler 212. The upper electrode 15 is electrically coupled to the power supply electrode 211 by the conductive material member 213. The conductive material member 213 is provided on the substrate 21 so as to extend across the upper electrode 15 and the power supply electrode 211 and is formed of conductive material. With this configuration, the upper electrode 15 is supplied with the sensor power supply signal from the power supply circuit 123 via the power supply electrode 211.

A second wiring line 260 is electrically coupled to the power supply electrode 211 by the conductive material member 213. The second wiring line 260 is formed, for example, of metal wiring, and is formed of conductive material. The second wiring line 260 is formed of material having better conductivity than the upper electrode 15. The second wiring line 260 is provided in a layer between the uppermost layer side of the substrate 21 and the optical sensor PD in the third direction Dz. The second wiring line 260 is electrically coupled to the upper electrode 15 and the coupler 212. The second wiring line 260 is coupled to the control circuit 122 via a wiring line 261 that supplies a power supply voltage to the second wiring line 260.

The control circuit 122 is a circuit that supplies control signals to the optical sensor PD to control detection operations thereof. Each of the optical sensors PD outputs, to the detection circuit 48, an electrical signal corresponding to the light emitted thereto as a detection signal.

As illustrated in FIG. 4, the lowermost layer of the substrate 21 includes anode coupling wiring 23 and cathode coupling wiring 24. The lowermost layer of the substrate 21 is provided with through-holes TH1 and TH2 that pass therethrough in the third direction Dz. One end of the anode coupling wiring 23 is coupled to the anode sides of the first light source 61, the second light source 62, and the third light sources 63 and 64 via the through-hole TH1. One end of the cathode coupling wiring 24 is coupled to the cathode sides of the first light source 61, the second light source 62, and the third light sources 63 and 64 via the through-hole TH2. The other end of each of the anode coupling wiring 23 and the cathode coupling wiring 24 is coupled to the control circuit 122. The first light source 61, the second light source 62, and the third light sources 63 and 64 are switched between lit and unlit states based on control signals from the control circuit 122.

As illustrated in FIGS. 3 and 5, the detection device 100 further includes a first light-blocking wall 55 and a second light-blocking wall 56. The first light-blocking wall 55 is provided for each of the first opening OP1, the second opening OP2, and the third opening OP3. The first light-blocking walls 55 respectively surrounds the first light source 61, the second light source 62, and the third light sources 63 and 64, to block light incident on the first light source 61, the second light source 62, and the third light sources 63 and 64. The first light-blocking walls 55 protrude higher than each of the first light source 61, the second light source 62, and the third light sources 63 and 64 in the third direction Dz.

The second light-blocking wall 56 is positioned at the outer periphery of the optical sensor PD. The second light-blocking wall 56 blocks light incident on the optical sensor PD. The second light-blocking wall 56 protrudes higher than the optical sensor PD in the third direction Dz. The first and the second light-blocking walls 55 and 56 are formed of black resin or metal material such as aluminum. The first light-blocking walls 55 can hinder light emitted from the first light source 61, the second light source 62, and the third light sources 63 and 64 from directly entering the optical sensor PD. The second light-blocking wall 56 can hinder external light from entering the optical sensor PD.

As illustrated in FIG. 5, the detection device 100 further includes a protective member 80 and a projection 81. The protective member 80 and the projection 81 are formed of light-transmitting resin.

The projection 81 projects in the third direction Dz. The first and the second light sources 61 and 62 are covered by the projection 81. This configuration allows the projection 81 to be easily pushed toward the object to be detected Fg when coming into contact with the object to be detected Fg, thus making it easier for light to enter the object to be detected Fg.

The protective member 80 is provided in contact with the tops of the first and the second light-blocking walls 55 and 56in the third direction Dz. The optical sensor PD, the first light source 61, the second light source 62, and the third light sources 63 and 64 are covered by the protective member 80. This configuration can prevent the object to be detected Fg from contacting the optical sensor PD, the first light source 61, the second light source 62, and the third light sources 63 and 64. The protective member 80 may be formed integral with the projection 81.

As illustrated in FIG. 6, the detection device 100 includes the substrate 21, an insulating layer 27, and the optical sensor PD.

The insulating layer 27 is provided on the uppermost layer of the substrate 21. The insulating layer 27 is provided between the substrate 21 and the optical sensor PD. The insulating layer 27 may be an inorganic insulating film or an organic insulating film.

The optical sensor PD is provided on the insulating layer 27. The optical sensor PD includes the lower electrode 11, a lower buffer layer 12, an active layer 13, an upper buffer layer 14, and the upper electrode 15. In the optical sensor PD, the lower electrode 11, the lower buffer layer 12 (hole transport layer), the active layer 13, the upper buffer layer 14 (electron transport layer), and the upper electrode 15 are stacked in this order in the third direction Dz orthogonal to the substrate 21.

The lower electrode 11 is an anode electrode of the optical sensor PD, and is formed, for example, of light-transmitting conductive material such as indium tin oxide (ITO). The active layer 13 changes in characteristics (for example, voltage-current characteristics and resistance value) depending on light emitted thereto. Organic material is used as the material of the active layer 13. Specifically, the active layer 13 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 13, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 13 can be formed by a vapor deposition process (dry process) using any of the low-molecular-weight organic materials listed above. In this case, the active layer 13 may be, for example, a multilayered film of CuPc and F₁₆CuPc, or a multilayered film of rubrene and C₆₀. The active layer 13 can also be formed by a coating process (wet process). In this case, the active layer 13 is made using material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The active layer 13 can be a film made of a mixture of P3HT and PCBM, or a film made of a mixture of F8BT and PDI.

The lower buffer layer 12 is a hole transport layer. The upper buffer layer 14 is an electron transport layer. The lower buffer layer 12 and the upper buffer layer 14 are provided to facilitate holes and electrons generated in the active layer 13 to reach the lower electrode 11 or the upper electrode 15. The lower buffer layer 12 (hole transport layer) is in direct contact with the top of the lower electrode 11 and is also provided in an area between the adjacent lower electrodes 11. The active layer 13 is in direct contact with the top of the lower buffer layer 12. The material of the hole transport layer is a metal oxide layer. Tungsten oxide (WO₃), molybdenum oxide, or the like is used as the metal oxide layer.

The upper buffer layer 14 (electron transport layer) is in direct contact with the top of the active layer 13, and the upper electrode 15 is in direct contact with the top of the upper buffer layer 14. Polyethylenimine ethoxylated (PEIE) is used as the material of the electron transport layer.

The materials and the manufacturing methods of the lower buffer layer 12, the active layer 13, and the upper buffer layer 14 are merely exemplary, and other materials and manufacturing methods may be used. For example, each of the lower buffer layer 12 and the upper buffer layer 14 is not limited to a single-layer film and may be formed as a multilayered film that includes an electron blocking layer and a hole blocking layer.

The upper electrode 15 is provided on the upper buffer layer 14. The upper electrode 15 is a cathode electrode of the optical sensor PD, and is continuously formed over the entire optical sensors PD. In other words, the upper electrode 15 is continuously provided on the optical sensors PD. The upper electrode 15 faces the lower electrodes 11 with the lower buffer layer 12, the active layer 13, and the upper buffer layer 14 interposed therebetween. The upper electrode 15 is formed, for example, of light-transmitting conductive material such as ITO or indium zinc oxide (IZO). A portion of an end of the upper surface of the upper electrode 15 is electrically coupled to the conductive material 213.

### First Operation Example of Detection Device

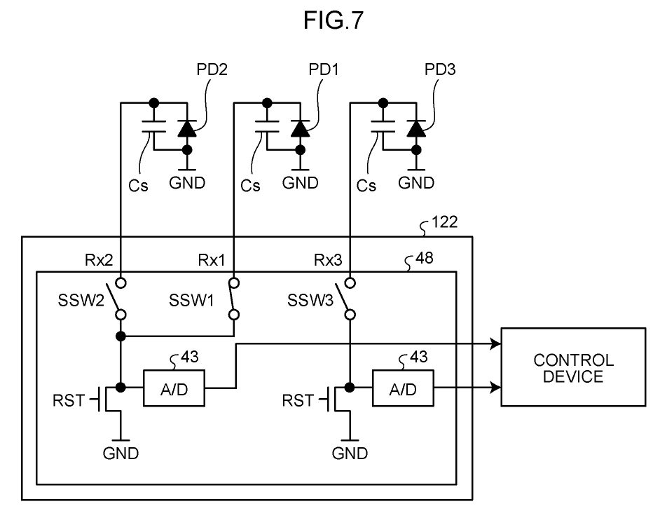

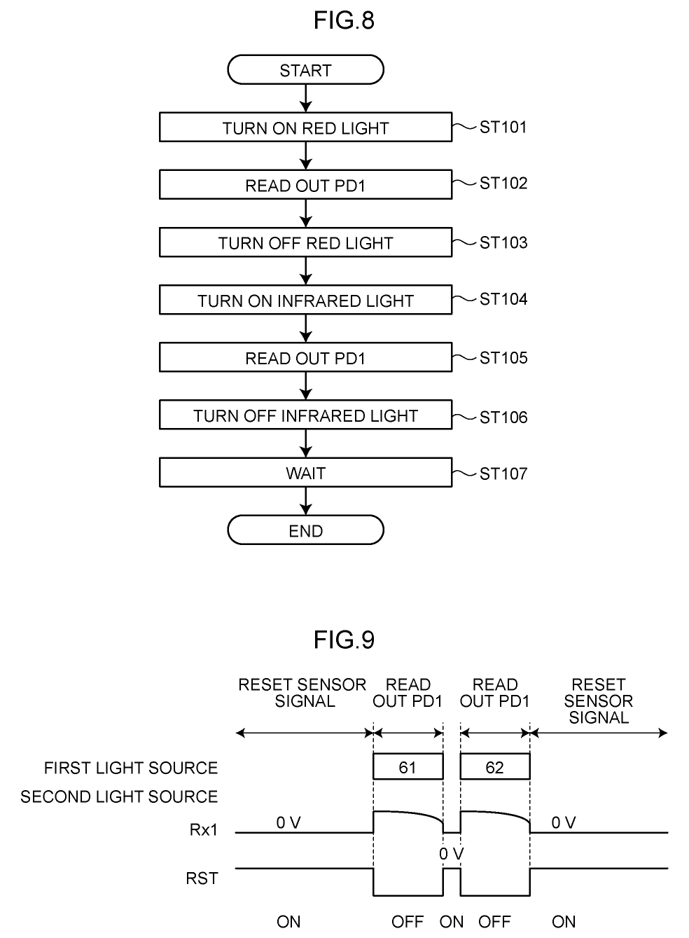

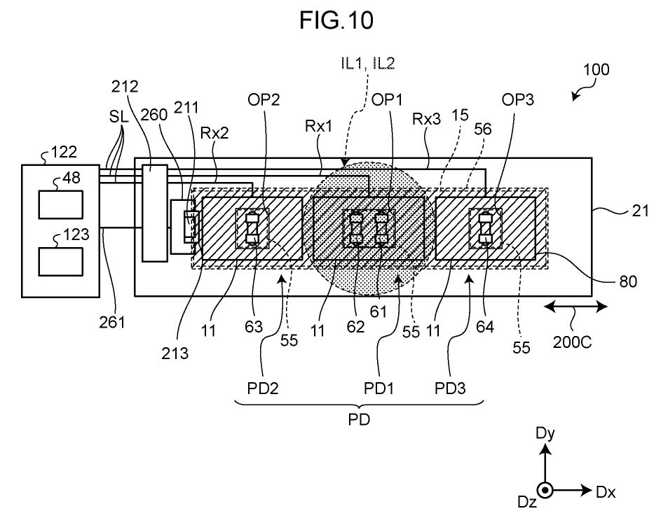

The following describes a first operation example in which the detection device 100 calculates an oxygen saturation level in blood (hereinafter, referred to as a "blood oxygen saturation level (SpO₂)"). FIG. 7 is a circuit diagram illustrating a configuration example of the detection device according to the first embodiment. FIG. 8 is a flowchart illustrating an example of a process to output the blood oxygen saturation level in the first embodiment. FIG. 9 is a timing waveform diagram illustrating an operation example using the first light source and the second light source of the detection circuit according to the first embodiment. FIG. 10 is a plan view illustrating a pulse wave detection area when using the first light source and the second light source of the detection device according to the first embodiment.

As illustrated in FIG. 7, the detection circuit 48 includes switch circuits SSW1, SSW2, and SSW3, reset transistors RST, and analog-to-digital (A/D) conversion circuits 43.

The first wiring lines Rx1, Rx2, and Rx3 are coupled to the detection circuit 48. The detection circuit 48 is electrically coupled to the lower electrodes 11. The detection circuit 48 may be formed as a separate circuit from the control circuit 122.

Holding capacitance Cs is generated between the anode side of each of the first optical sensor PD1, the second optical sensor PD2, and the third optical sensor PD3 and a corresponding one of the first wiring lines Rx1, Rx2, and Rx3.

The anode sides of the optical sensors PD are coupled to GND. The switch circuits SSW1, SSW2, and SSW3 are coupled to the cathode sides of the optical sensors PD via the first wiring lines Rx1, Rx2, and Rx3.

One of the source and the drain of each of the reset transistor RST is coupled to any one of the switch circuits SSW1, SSW2, and SSW3 and one of the A/D conversion circuits 43. The other of the source and the drain of each of the reset transistors RST is coupled to GND. When the reset transistor RST is brought into the on state (conducting state), the cathode potential of the optical sensor PD is reset, and the sensor signal is reset.

The reset transistors RST have a single-gate structure, but may have what is called a double-gate structure configured by coupling two transistors in series, or may have a configuration in which three or more transistors are coupled in series.

One of the A/D conversion circuits 43 converts analog signals output from the first optical sensor PD1 into digital signals. An externally located control device performs signal processing on the digital signals from the A/D conversion circuit 43.

In the first operation example of the detection device 100, the switch circuit SSW1 is in the on state (refer to FIG. 7). Assume that an initial state is a state in which the reset transistor RST is in the on state and the sensor signal is reset (refer to FIG. 9).

As illustrated in FIG. 8, the first light source 61 is turned on, and the red light is emitted to the object to be detected Fg (Step ST101). At Step ST101, the reset transistor RST is in the off state (refer to FIG. 9).

Then, red light IL1 (refer to FIG. 10) reflected by the object to be detected Fg illuminates the first optical sensor PD1, and the first optical sensor PD1 is read out (Step ST102, refer to FIG. 9).

Then, the first light source 61 is turned off to stop emitting the red light IL1 to the first optical sensor PD1 (Step ST103). At Step ST103, the reset transistor RST is in the on state (refer to FIG. 9).

Then, the second light source 62 is turned on to emit the infrared light to the object to be detected Fg (Step ST104). At Step ST104, the reset transistor RST is in the off state (refer to FIG. 9).

Then, infrared light IL2 (refer to FIG. 10) reflected by the object to be detected Fg illuminates the first optical sensor PD1, and the first optical sensor PD1 is read out (Step ST105, refer to FIG. 9).

Then, the second light source 62 is turned off to stop emitting the infrared light IL2 to the first optical sensor PD1 (Step ST106). At Step ST106, the reset transistor RST is in the on state (refer to FIG. 9).

The first and second light sources 61 and 62 are in a static lighting (direct-current (DC) lighting) state while being on, and the light is always irradiating the object to be detected Fg. In the process up to Step ST106, the first and the second light sources 61 and 62 are on while the third light sources 63 and 64 are not on. When the process up to Step ST106 has ended, the system is brought into a waiting state (Step ST107), and the first optical sensor PD1 measures pulse wave data. The externally provided control device calculates the blood oxygen saturation level (SpO₂) from the pulse wave data. The process for outputting the blood oxygen saturation level (SpO₂) then ends. The blood oxygen saturation level (SpO₂) refers to a ratio of an amount of oxygen actually bound to hemoglobin to the total amount of oxygen under the assumption that the oxygen is bound to all the hemoglobin in the blood. When acquiring the blood oxygen saturation level (SpO₂), for example, a pulse wave acquired using the red light and a pulse wave acquired using the infrared light are used. When acquiring the blood oxygen saturation level (SpO₂), for example, a vein image acquired using the red light and a vein image acquired using the infrared light may also be used.

### Second Operation Example of Detection Device

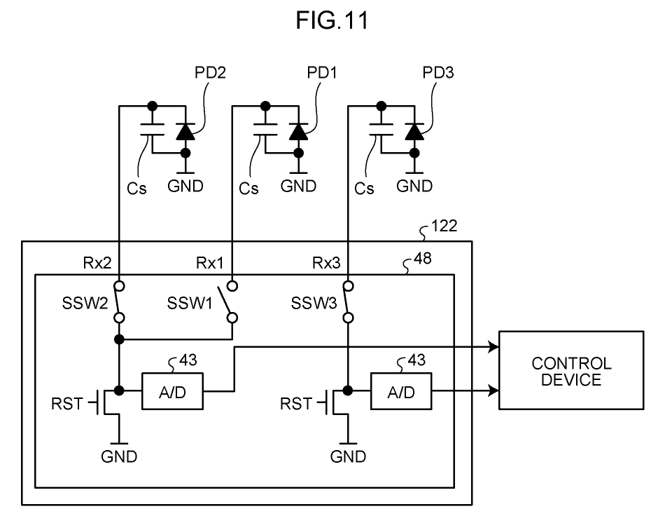

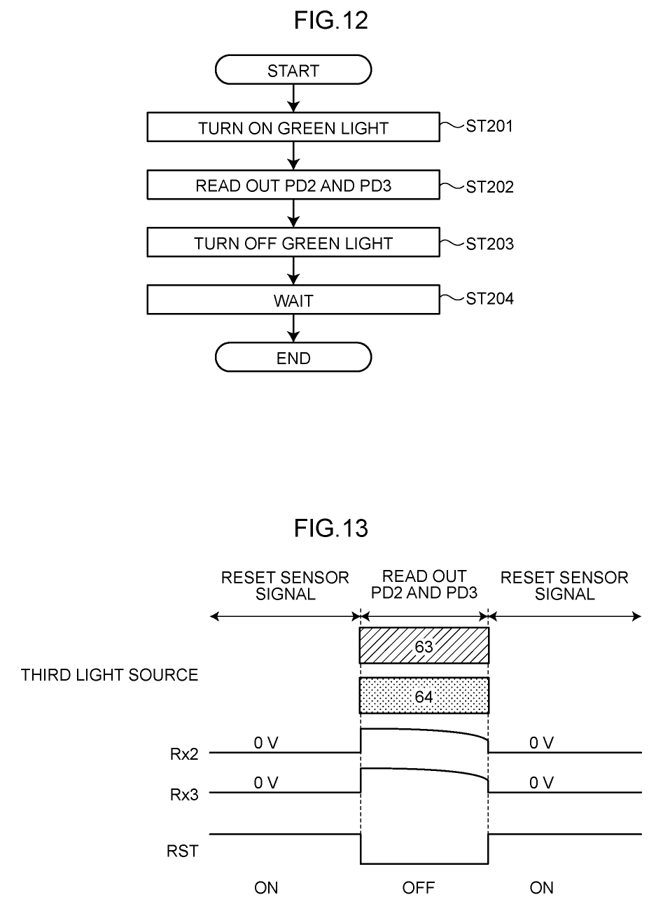

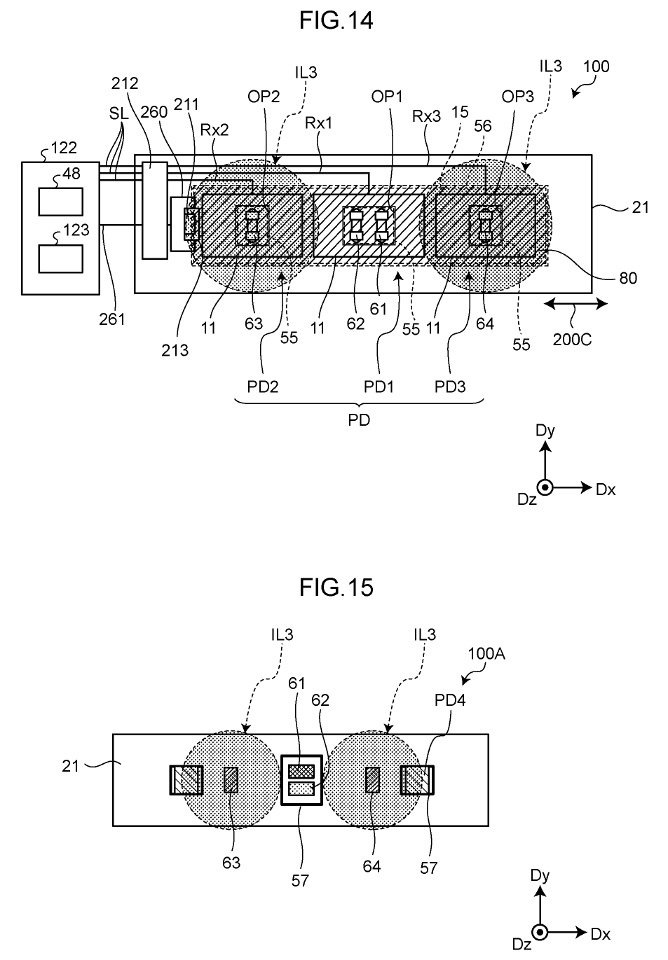

The following describes a second operation example in which the detection device 100 calculates a heart rate. FIG. 11 is the circuit diagram illustrating a configuration example of the detection device according to the first embodiment. FIG. 12 is a flowchart illustrating exemplary processing related to output of the heart rate in the first embodiment. FIG. 13 is a timing waveform diagram illustrating an operation example using the third light sources of the detection circuit according to the first embodiment. FIG. 14 is a plan view illustrating pulse wave detection areas when using the third light sources of the detection device according to the first embodiment. The components in FIG. 11 is the same as those in FIG. 7, and therefore, will not be described.

In the second operation example of the detection device 100, the switch circuits SSW2 and SSW3 are in the on state (refer to FIG. 11). Assume that an initial state is a state in which each of the reset transistors RST is in the on state and the sensor signal is reset (refer to FIG. 13).

As illustrated in FIG. 12, the third light sources 63 and 64 are turned on, and the green light is emitted to the object to be detected Fg (Step ST201). At Step ST201, each of the reset transistors RST is in the off state (refer to FIG. 13).

Then, green light IL3 (refer to FIG. 14) reflected by the object to be detected Fg illuminates the second and the third optical sensors PD2 and PD3, and the second and the third optical sensors PD2 and PD3 are read out (Step ST202, refer to FIG. 13).

Then, the third light sources 63 and 64 are turned off to stop emitting the green light to the second and the third optical sensors PD2 and PD3 (Step ST203). At Step ST203, each of the reset transistors RST is in the on state (refer to FIG. 13).

The third light sources 63 and 64 are in the static lighting (DC lighting) state while being on, and the light is always irradiating the object to be detected Fg. In the process up to Step ST203, the third light sources 63 and 64 are on while the first and the second light sources 61 and 62 are not on. When the process up to Step ST203 has ended, the system is brought into a waiting state (Step ST204), and the second and the third optical sensors PD2 and PD3 each measure the pulse wave data. The externally provided control device calculates the heart rate from the pulse wave data. The process for outputting the heart rate then ends.

FIG. 15 is a plan view illustrating pulse wave detection areas of a detection device according to a comparative example. As illustrated in FIG. 15, a detection device 100A according to the comparative example includes optical sensors PD4, the first light source 61, the second light source 62, the third light sources 63 and 64, and light-blocking walls 57, on the substrate 21. The optical sensors PD4, a pair of the first light source 61 and the second light source 62 are surrounded by the light-blocking walls 57, respectively.

In the detection device 100A according to the comparative example, the optical sensor PD4, the first light source 61, the second light source 62, and the third light sources 63 and 64 are horizontally arranged on the substrate 21, as compared with in the detection device 100.

In the detection device 100A according to the comparative example, the green light IL3 emitted from each of the third light sources 63 and 64 to the object to be detected Fg and reflected by the object to be detected Fg irradiates only part of the corresponding optical sensor PD4. Therefore, each of the optical sensor PD4 can absorb only a limited amount of light, leaving room for improvement in light use efficiency.

In contrast thereto, the detection device 100 according to the first embodiment can detect the pulse waves by emitting the red light IL1 and the infrared light IL2, each of which is reflected by the object to be detected Fg and illuminates the entire first optical sensor PD1 as illustrated in FIG. 10, thereby improving the light use efficiency.

The detection device 100 according to the first embodiment can also detect the pulse waves by emitting the green light IL3, which is reflected by the object to be detected Fg and illuminates the entire areas of the respective second and third optical sensors PD2 and PD3 as illustrated in FIG. 14, thereby improving the light use efficiency.

In the detection device 100A according to the comparative example, a distance between each of the optical sensors PD4 and an area in which the first light source 61, the second light source 62, and the third light sources 63 and 64 are mounted is long.

In contrast thereto, in the detection device 100 according to first embodiment, the multiple openings OP penetrating the optical sensor PD are provided, and a pair of the first light source 61 and the second light source 62, the third light sources 63 and 64 are respectively arranged in the openings OP. Therefore, the distance between the light sources and the area for mounting the components of the photodiodes can be reduced, and the size of the entire detection device 100 can be reduced.

While the preferred embodiment has been described above, the present invention is not limited to such an embodiment. The content disclosed in the embodiment is merely an example and can be variously modified within the scope not departing from the gist of the present invention. Any modifications appropriately made within the scope not departing from the gist of the present invention also naturally belong to the technical scope of the present invention. At least one of various omissions, substitutions, and changes of the components can be made without departing from the gist of the embodiment described above and the modifications thereof.

What is claimed is:

1. A detection device comprising:
a substrate;
a light source configured to emit light to an object to be detected; and
an organic photodiode in which a lower electrode, a lower buffer layer, an active layer, an upper buffer layer, and an upper electrode are stacked in the order as listed, wherein
the organic photodiode is provided on the substrate,
an opening provided so as to penetrate the organic photodiode exposes a surface of the substrate, and
the light source is provided on the substrate in the opening.

2. The detection device according to claim 1, wherein
a plurality of the lower electrodes are provided at different positions,
the lower buffer layer, the active layer, the upper buffer layer, and the upper electrode are stacked on each of the lower electrodes,
a plurality of the openings are provided through the respective lower electrodes, and
a plurality of the light sources are provided in the respective openings.

3. The detection device according to claim 2, wherein
each of the openings is provided with a first light-blocking wall that surrounds a corresponding one of the light sources and is configured to block light incident on the light source, and
the first light-blocking wall protrudes in a direction orthogonal to the substrate and is higher than the light source.

4. The detection device according to claim 3, wherein
an outer periphery of the organic photodiodes is provided with a second light-blocking wall configured to block light incident on the organic photodiodes, and
the second light-blocking wall protrudes in the direction orthogonal to the substrate and is higher than the organic photodiodes.

5. The detection device according to claim 4, wherein
the openings comprise a first opening, a second opening, and a third opening,
the light sources comprise a first light source configured to emit red light, a second light source configured to emit infrared light, and third light sources configured to emit green light,
the first opening is provided with the first light source and the second light source,
the second opening and the third opening are respectively provided with the third light sources, and
the first opening is provided between the second opening and the third opening.

6. The detection device according to claim 5, wherein the first light source and the second light source are covered by a projection that has a light-transmitting property and projects in the direction orthogonal to the substrate.

7. The detection device according to claim 5, wherein the organic photodiodes and the third light sources are covered by a light-transmitting protective member.

8. The detection device according to claim 7, wherein
the third light sources are configured to be on while the first light source and the second light source are not on, and
the first light source and the second light source are configured to be on while the third light sources are not on.

9. The detection device according to claim 8, wherein a control device is externally provided, wherein
the control device is configured to
calculate a blood oxygen saturation level from data measured while the first light source and the second light source are on and
calculate a heart rate from data measured while the third light sources are on.
